(19) United States
(12) Patent Application Publication
(10) Pub. No.: US 2025/0360073 A1
(43) Pub. Date: Nov. 27, 2025

(54) SOLUBILIZING PIGMENTS EXTRACTED FROM FUNGI FOR APPLICATIONS SUCH AS COSMETIC PRODUCTS

(71) Applicant: Jesse Adler, Farmington Hills, MI (US)

(72) Inventor: Jesse Adler, Farmington Hills, MI (US)

(21) Appl. No.: 18/874,349

(22) PCT Filed: Jun. 13, 2023
(86) PCT No.: PCT/US23/25129

§ 371 Date: Dec. 12, 2024

Related U.S. Application Data
(60) Provisional application No. 63/351,564, filed on Jun. 13, 2022.

(51) Int. Cl. A61K 8/9728; A61Q 1/02; A61Q 1/06; A61Q 1/10; A61Q 1/12

(52) U.S. Cl. CPC A61K 8/9728; A61Q 1/02; A61Q 1/06; A61Q 1/10; A61Q 1/12; A61K 2800/43

(57) ABSTRACT

A method of and colorant from solubilizing pigments extracted from fungi in natural oils or natural solvents, where the colorant is useful for providing color or tint to cosmetic products. The pigments may be extracted from mushroom, lichen, mold, yeast, or a combination, upon mixing with a solvent and filtering and purifying the mixture to produce pure pigment in solvent. The pure pigment in solvent is added to an oil and the solvent is volatilized away. The pure pigment and oil may be used in cosmetic products, such as lipstick, eyeshadow, mascara, moisturizer, and sunscreen.

### CLAIM OF BENEFIT OF FILING DATE

The present application claims the benefit of the filing date of U.S. Provisional Application No. 63/351,564, filed on Jun. 13, 2022, the contents of which are hereby incorporated by reference in their entirety for all purposes.

### FIELD

The present teachings generally relate to solubilizing pigments extracted from fungi, the use of pigments extracted from fungi, or both, and more particularly to the solubilizing pigments extracted from fungi in natural oils or natural solvents, use of the pigments extracted from fungi in cosmetic products, or both.

### BACKGROUND

Color is a vital feature of life and as such, humans have been on a quest to harness the boldest and brightest colors for thousands of years, originally creating pigments from natural materials, such as clay and ash, until the discovery of synthetic colorants in the mid-19th century.

The advent of synthetic colorants triggered a new wave of colorful possibilities, with hues and performance characteristics well-beyond that of colorants from plants, animals, and minerals. However, synthetic colorants are made from fossil fuels (a non-renewable resource) and some have been found to have harmful effects on both humans (as carcinogens) and the planet (as pollutants). Growing awareness of these issues has increased the global demand for alternative renewable sources of color in food, cosmetic, and textile sectors.

Certain colors are difficult to produce or difficult to keep stable. Natural colors, especially, may be highly unstable. As the colorant is mixed with other components or substances of a particular pH or heated or exposed to light, the color may change drastically. Temperature, presence of oxygen, light, or a combination thereof, are known to break down natural colors. In baking, for example, batter for a red velvet cake has a significantly different color from a baked red velvet cake. This may be due, at least in part, to the instability of the red colorant when exposed to the baking temperatures. These stability complications are especially true in red hues or shades, which are not only desirable in certain foods or textiles but are also desirable in beauty products such as lip products (e.g., lipstick, lip gloss), cheek colorant (e.g., blush), and nail polish.

Therefore, there remains a need for creating color from renewable and/or natural sources. There also remains a need for alternative coloring sources as users have begun to question the provenance of the pigments/colorants they use on their skin every day. There is also a need for producing stable colorants for use in applications such as cosmetics. There is also a need for the cosmetics themselves to remain the desired color once the cosmetics have been produced.

### SUMMARY

The present teachings relate to a colorant and methods of producing the colorant. The colorant may be useful for providing color or tint to a cosmetic product. The present teachings include a colorant with one or more pigments derived from one or more fungi. The colorant may be used for coloring a cosmetic product, such as a lipstick, eyeliner, eyeshadow, mascara, blush, bronzer, tinted moisturizer, self-tanner, foundation, nail polish, or sunscreen.

The colorant may include pigment and one or more oils. The oil may include castor oil, almond oil, sunflower oil, walnut oil, linseed oil, Danish oil, MCT oil, vegetable glycerin, or a combination thereof.

The colorant may include pigment extracted from mushroom, lichen, mold, yeast, or a combination thereof. The pigment may be extracted from spalting fungi. The pigment may be selected from one or more of isoprenoids, polyketides, quinones, naphthoquinones, flavins, pyrans, pyran-related, pyrones, laetiporic acids, or other non-proteins. The pigments may include any of the pigments listed in Table 2, alone or in combination. For example, pigments may include beta carotene, melanin, astaxanthin, xylindein, draconin, unnamed yellow/purple pigment, monascus pigments, parietin, or a combination thereof.

The colorant may include pigments extracted from one or more of the following, where the organism is in italics, and the pigment and color are in parentheses: Chlorociboria aeruginascens (Xylindein; blue-green), Chlorociboria aeruginosa (Xylindein; blue-green), Scytalidium cuboideum (Draconin; red), Monascus purpureus (Rubropunctatine, Monascorubrine, Monascoflavin, Lovastatin (mevinolin; orange), Monascus purpureus (Ankaflavin, Mosascin; yellow), Scytalidium ganodermophthorum (unknown pigment name; yellow, olive), Scytalidium ganodermophthorum (unknown pigment name; purple), Inonotus obliquus (Eumelanin; brown/black), Inonotus obliquus (Pheomelanin; red/yellow), Xanthoria parietina (Parietin; orange), Xanthophyllomyces dendrorhous (Astaxanthin; orange), Blakeslea trispora (Beta-carotene; yellow).

The pigment may be extracted from a fungus via solubilization with a solvent. The solvent may include dichloromethane, water, acetone, methanol, ethanol, acetonitrile, chloroform, tetrahydrofuran (THF), pyridine, N,N-dimethylformamide, hexane, or a combination thereof. The fungus may be dried and/or ground into a powder. The powdered fungus may be mixed with the solvent. The solvent and powdered fungus may be filtered or separated to produce crude pigment in solvent. One or more purification steps may occur to produce purified pigment in solvent.

The purified pigment in solvent may be added to one or more oils. The oil may include castor oil, almond oil, sunflower oil, walnut oil, linseed oil, Danish oil, MCT oil, vegetable glycerin, or a combination thereof. The solvent may be volatilized or evaporated to produce a liquid oil-based pigment dispersion that can be mixed with a cosmetic base to produce the final cosmetic product.

Therefore, the present teachings provide a natural, sustainable colorant and method for producing the colorant. The colorant may be used to provide color or tint to products, including but not limited to cosmetic products.

### DETAILED DESCRIPTION

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

The present teachings relate generally to methods and compositions associated with the solubilizing of pigments extracted from organic and/or inorganic sources, such as fungi, in solvent. Solvents may include organic solvents (e.g., a carbon-based solvent, such as dichloromethane), inorganic solvents (e.g., a solvent free of or substantially free of carbon, water), natural oils, or a combination thereof. It is also possible to employ supercritical fluid extraction (SFE) with CO₂. SFE may be used in combination with or as an alternative to other solvent extraction methods. The present teachings also relate to methods and compositions associated with utilizing or implementing these pigments in cosmetic products.

A cosmetic is a product intended to be applied to the human body for such purposes as cleansing, beautifying, promoting attractiveness, or altering appearance. A cosmetic product may be applied to hair, skin, nails, parts of the face (e.g., eyelids, lips, cheeks), or a combination thereof. Cosmetics may include cleansers such as body wash, shower gel, bubble bath, scrubs, bar soap, and the like. Cosmetics may include beauty products such as makeup. Cosmetics may include lotions, creams, gels, liquids, solids, sprays, powders, the like, or a combination thereof. Cosmetics may include products applied to the nails, such as nail polish.

Cosmetics may be applied and/or dispensed in any suitable fashion. For example, cosmetics may be applied with or as a brush, pencil, crayon, stick, pouf, sponge, fingers or hand, applicator, cloth, wand, loofah, rollerball, spray bottle (e.g., atomizer, can, aerosol can, trigger-style spray bottle), pump-style dispenser, directly from the container (e.g., a tube, such as a tube of lipstick or tube of deodorant), the like, or a combination thereof.

Cosmetics may be contained within any suitable container. Cosmetics may be contained within and/or applied via tube (e.g., a squeeze tube, tube and wand and/or brush applicator, a lipstick tube, a casing). Cosmetics may be contained within a bottle. The cosmetics in the bottle may be dispensed by spraying, squeezing, pumping, pouring, scooping, rubbing, the like, or a combination thereof. The cosmetics may be in a container such as a pot or jar. The cosmetics may be in a container such as a compact or clamshell-type container. The cosmetics may be in a container made of any suitable material, including but not limited to glass, plastic, recycled materials, cardboard, paper, 3D-printed material, or a combination thereof. The cosmetic may be free of a container. The container may be removable (e.g., removable paper, cardboard, and/or plastic packaging).

Cosmetics may include products to be used on or around the eye area, such as, but not limited to, eye cream, eye primer, eye brightener, under-eye products (e.g., concealer), eye shadow (e.g., powder eye shadow, cream eye shadow, liquid eye shadow), eye liner (e.g., eye liner pencil, eye liner crayon, liquid eye liner), mascara, eyebrow products (e.g., pencil, powder, gel), and the like.

Cosmetics may include products to be used on or around the lips, such as, but not limited to, lipstick, lip gloss, lip crayon, liquid lip color, lip moisturizer, lip balm, lip exfoliator, lip liner (e.g., lip liner pencil, lip liner crayon, liquid lip liner), the like, or a combination thereof. The lip product may, for example, be a solid, liquid, gel, or a combination thereof.

Cosmetics may include products to be used on the skin, such as to improve or alter skin texture (e.g., smoothing, altering pore size or pore appearance), camouflage imperfections (e.g., blemishes, wrinkles, dark spots, redness), moisturize, heal, reduce or prevent sun exposure or damage (e.g., via sunscreen), alter skin tone appearance (e.g., even skin tone, darken or lighten skin tone, add color or shimmer), the like, or a combination thereof. Skin products may include, but are not limited to, lotions, creams, gels, powders, solids (e.g., those applied by rubbing the solid on the skin, an applicator, or both), sunscreens, moisturizers (e.g., tinted moisturizer), self-tanner, or skin lightener. Skin products may include makeup such as concealer, foundation, pressed powder, loose powder, color corrector, blush, bronzer (e.g., powder, gel, liquid, stick), highlighter, contour makeup, the like, or combination thereof.

Cosmetics may include products for hair and/or nails. Hair products may include shampoos, conditioners, leave-in treatments, wash-out treatments, spray products, dyes (e.g., permanent, semi-permanent, or washable), the like, or a combination thereof. Nail products may include nail polish, gel polish, nail varnish, nail enamel, nail dip, the like, or a combination thereof.

The cosmetics and/or their effects may be washable, such that they are easily removed (e.g., via water, washcloth, wipe, oil, or the like). The cosmetics and/or their effects may be semi-permanent (e.g., wearing away over a period of time). The cosmetics and/or their effects may be removable with certain substances, such as substances other than water (e.g., nail polish remover).

Cosmetics may include one or more colorants for providing a desired look to the product, providing a desired look in the container, providing a desired look on the user, or combination thereof. A colorant may provide color to the product. Colorant may include one or more pigments, one or more solvents, one or more oils, or a combination thereof. One or more ingredients may be removed, filtered out, separated, evaporated (totally or partially), or a combination thereof, prior to mixing the colorant with other components (e.g., a cosmetic base) to make the final product. Color may be provided, at least in part, by one or more pigment types.

Cosmetics may include one or more ingredients for providing shimmer, sparkle, luster, sheen, pearlescence, or a combination thereof, which may be included within or part of the colorant or may be separate from the colorant.

Colors (e.g., colors of the pigments or derived from the pigments, colors of or created by the colorant) may include, but are not limited to, black, black-brown, blue, blue-green, blue-yellow, brown, brown-red, cinnamon brown, dark, deep red, goldenrod, gray, green-yellow, orange, orange-red, pink, pink-red, purple, red, red fluorescent, yellow, yellow-orange, yellow-red, colors therebetween, or a combination thereof. Colors may be mixed to create other colors. For example, mixing a yellow and a blue may produce a green color.

One or more pigments may be derived from or extracted from natural sources. Natural sources may include fungi. Natural sources may include mushrooms, lichens, molds, and yeasts. A mushroom is the spore-bearing fruiting body of a fungus. A lichen is a composite organism including one or more photobiont (photosynthetic organisms such as algae or cyanobacteria) and one or more mycobiont (fungal organisms). Molds are structures of multicellular filaments that fungi can form in the process of digesting a substrate. Molds may include saprotrophic fungi which digest organic matter, such as spalting fungi. Yeasts are unicellular fungi that do not produce hyphae and reproduce asexually by mitosis. When discussing fungus or fungi herein, it is contemplated that this may encompass a single fungus or two or more fungi.

The fungus or fungi may be from the genus, but are not limited to, Agaricus, Albatrellus, Aleuria, Alternaria, Aschersonia, Ascochyta, Agaricus, Albatrellus, Aleuria, Alternaria, Aschersonia, Ascochyta, Ashbya, Aspergillus, Batrachochytrium, Blakeslea, Boletus, Botrytis, Candida, Cantharellus, Cercospora, Chaetomium, Chlorociboria, Chroogomphus, Cladosporium, Claviceps, Cochliobolus, Colletotrichum, Cordyceps, Cortinarius, Cryptococcus, Curvularia, Cylindrocarpon, Dermocybe, Drechslera, Elsinoe, Emericella, Epicoccum, Epidermophyton, Evernia, Fomes, Fusarium, Gomphidius, Helminthosporium, Herpotrichia, Inonotus, Isaria, Kluyveromyces, Lactarius, Laetiporus, Magnaporte, Melanogaster, Mevinolin, Microsporum, Monascus, Mucor, Mycena, Nectria, Neurospora, Paramelia, Penicilliopsis, Penicillium, Pestalotia, Phaeococcomyces, Phaffia, Phoma, Phomopsis, Phycomyces, Phyllosticta, Physcia, Pichia, Platismatia, Pseudomonas, Pycnoporus, Pyricularia, Pyronema, Ramalina, Ramularia, Rhodosporidium, Rhodotorula, Saccharomyces, Schizophyllum, Sclerotinia, Sclerotium, Scytalidium, Sporidiobolus, Sporidiobolus, Sporobolomyces, Stemphylium, Streptomyces, Suillus, Talaromyces, Thielaviopsis, Trametes, Trichoderma, Trichophyton, Usnea, Ustilago, Verticillium, Xanthophyllomyces, Xanthoria, Xylaria, Yarrowia, or any combination thereof.

The species of fungus or fungi may include, but is not limited to, the following in Table 1, alone or in any combination.

TABLE 1
Genus and Species Names

| Genus Name | Species Name |
|---|---|
| Agaricus | bisporus |
| Albatrellus | caeruleoporus |
| Aleuria | aurantia |
| Alternaria | tagetica |
| Alternaria | alternata |
| Aschersonia | aleyrodis |
| Ascochyta | rabiei |
| Ashbya | gossypii |
| Aspergillus | nidulans |
| Aspergillus | niger |
| Aspergillus | tamarii |
| Aspergillus | tubingensis |
| Aspergillus | flavus |
| Aspergillus | terreus |
| Aspergillus | giganteus |
| Aspergillus | sp. |
| Aspergillus | oryzae |
| Aspergillus | fonsecaeus |
| Aspergillus | versicolor |
| Aspergillus | glaucus |
| Aspergillus | cristatus |
| Aspergillus | repens |
| Aspergillus | sulphureus |
| Aspergillus | melleus |
| Batrachochytrium | dendrobatidis |
| Blakeslea | trispora |
| Boletus | sp. |
| Botrytis | cinerea |
| Candida | famata |
| Candida | lipolytica |
| Cantharellus | cinnabarinus |
| Cercospora | sp. |
| Cercospora | piaropi |
| Chaetomium | globosum |
| Chlorociboria | aeruginascens |
| Chlorociboria | aeruginosa |
| Chroogomphus | rutilus |
| Cladosporium | sp. |
| Cladosporium | herbarum |
| Claviceps | purpurea |
| Cochliobolus | miyabeanus |
| Colletotrichum | lindemuthianum |
| Colletotrichum | lagenarium |
| Cordyceps | unilateralis |
| Cordyceps | sp. |
| Cordyceps | bifusispora |
| Cortinarius | sp. |
| Cryptococcus | neoformans |
| Curvularia | lunata |
| Cylindrocarpon | sp. |
| Dermocybe | austroveneta |
| Dermocybe | sanguinea |
| Dermocybe | sp. |
| Drechslera | dictyoides |
| Drechslera | graminea |
| Drechslera | phlei |
| Drechslera | teres |
| Drechslera | triticirepentis |
| Drechslera | avenae |
| Elsinoe | fawcetti |
| Emericella | sp. |
| Epicoccum | nigrum |
| Epidermophyton | floccosum |
| Evernia | prunastri |
| Fomes | fomentarius |
| Fusarium | fujikuroi |
| Fusarium | sp. |
| Fusarium | solani |
| Fusarium | sporotrichioides |
| Fusarium | oxysporum |
| Gomphidius | glutinosus |
| Helminthosporium | catenarium |
| Helminthosporium | graminium |
| Herpotrichia | rhodosticta |
| Inonotus | obliquus |
| Isaria | farinosa |
| Kluyveromyces | marxianus |
| Lactarius | sp. |
| Lactarius | indigo |
| Lactarius | deliciosus |
| Lactarius | lilacinus |
| Laetiporus | sulphureus |
| Magnaporte | grisea |
| Melanogaster | broomeianus |
| Microsporum | cookei |
| Microsporum | sp. |
| Monascus | purpureus |
| Monascus | sp. |
| Monascus | ruber |
| Monascus | pilosus |
| Mucor | circinelloides |
| Mycena | sanguinolenta |
| Mycena | haematopus |
| Mycena | aurantiomarginata |
| Nectria | haematococca |
| Neurospora | crassa |
| Paramelia | saxatilis |
| Penicilliopsis | clavariaeformis |
| Penicillium | sp. |
| Penicillium | albidum |
| Penicillium | oxalicum |
| Penicillium | purpurogenum |
| Penicillium | citrinum |
| Penicillium | marneffei |
| Penicillium | chrysogenum |
| Penicillium | islandicum |
| Penicillium | fagi |
| Pestalotia | sp. |
| Phaeococcomyces | sp. |
| Phaffia | rhodozyma |
| Phoma | saccardo |
| Phoma | sorghina |
| Phoma | lingam |
| Phoma | wasabiae |
| Phoma | exigua |
| Phomopsis | vexans |
| Phycomyces | sp. |
| Phycomyces | blakesleeanus |
| Physcia | adscendens |
| Physcia | tenella |
| Phyllosticta | capitalensis |
| Pichia | guilliermondii |
| Platismatia | glauca |
| Pseudomonas | aeruginosa |
| Pycnoporus | sanguineus |
| Pyricularia | oryzae |
| Pyronema | omphalodes |
| Ramalina | fastigiata |
| Ramularia | collocygni |
| Rhodosporidium | toruloides |
| Rhodosporidium | diobovatum |
| Rhodotorula | glutinis |
| Rhodotorula | graminis |
| Rhodotorula | minuta |
| Rhodotorula | mucilaginosa |
| Rhodotorula | sp. |
| Rhodotorula | rubra |
| Saccharomyces | neoformans |
| Schizophyllum | commune |
| Sclerotinia | sclerotiorum |
| Sclerotium | rolfsii |
| Scytalidium | cuboideum |
| Scytalidium | ganodermophthorum |
| Sporidiobolus | sp. |
| Sporobolomyces | roseus |
| Sporobolomyces | johnsonii |
| Sporobolomyces | longiusculus |
| Sporobolomyces | salmonicolor |
| Sporobolomyces | patagonicus |
| Sporobolomyces | roseus |
| Stemphylium | lycopersici |
| Streptomyces | lavendulae |
| Streptomyces | chromofuscus |
| Streptomyces | aureofaciens |
| Suillus | grevillei |
| Suillus | bovinus |
| Talaromyces | atroroseus |
| Talaromyces | albobiverticillius |
| Talaromyces | purpurogenus |
| Talaromyces | minioluteus |
| Talaromyces | marneffei |
| Thielaviopsis | basicola |
| Trametes | versicolor |
| Trichoderma | reesei |
| Trichoderma | viride |
| Trichoderma | harzianum |
| Trichoderma | aureoviride |
| Trichoderma | virens |
| Trichophyton | violaceum |
| Usnea | subfloridana |
| Ustilago | maydis |
| Ustilago | sp. |
| Verticillium | dahliae |
| Xanthophyllomyces | dendrorhous |
| Xanthoria | parietina |
| Xylaria | euglossa |
| Yarrowia | lipolytica |

Exemplary fungi for producing pigments of desirable colors may include, but are not limited to, Chlorociboria aeruginascens (Xylindein; blue-green), Chlorociboria aeruginosa (Xylindein; blue-green), Scytalidium cuboideum (Draconin; red), Monascus purpureus (Rubropunctatine, Monascorubrine, Monascoflavin, Lovastatin (mevinolin; orange), Monascus purpureus (Ankaflavin, Mosascin; yellow), Scytalidium ganodermophthorum (unknown pigment name; yellow, olive), Scytalidium ganodermophthorum (unknown pigment name; purple), Inonotus obliquus (Eumelanin; brown/black), Inonotus obliquus (Pheomelanin; red/yellow), Xanthoria parietina (Parietin; orange), Xanthophyllomyces dendrorhous (Astaxanthin; orange), Blakeslea trispora (Beta-carotene; yellow).

The fungus or fungi may be provided in any suitable form. The fungus or fungi may be naturally occurring (e.g., found in nature). Fungus may be harvested. Fungus may be ethically harvested. The fungus or fungi may be grown (e.g., as a culture, in a lab or facility, or both). An organism may be grown in solid culture. An organism may be grown in submerged culture. Growing conditions may be selected to increase, accelerate, or optimize pigment production. A fungus may produce more than one pigment color. For example, depending on the conditions it is grown in, M. Purpureus may produce red, orange, and/or yellow pigments simultaneously in different proportions. Culture conditions may be selected to drive an increased production of one pigment over another.

The fungus or fungi may be dried. For example, the fungus or fungi may be dried in an agar plate or by freeze drying. The fungus may be dried by exposure to air, light (e.g., UV light), heat, or a combination thereof. The fungus may be dried by adding water to solubilize the pigments within the fungus and heating (e.g., baking in an oven) until the water is evaporated, and the dried pigment may be scraped out. The fungus or fungi may be sufficiently dry such that it can undergo grinding to produce a powder. Grinding may occur using any suitable tool for producing a fine powder. For example, grinding of a dried culture or sample may be performed using a rasp, mortar and pestle, or other method. Any method of producing a fine powder from a dried fungus is within the scope of the teachings herein.

A solvent may be added to the powdered fungus or fungi. The solvent may be non-carcinogenic, at least at particular dosages or concentrations. The solvent may be a renewable solvent. Any suitable solvent may be used that is capable of achieving a desired result. The solvent may be an inorganic solvent. The solvent may be an organic solvent. The solvent may be an alcohol. The solvent may be a ketone. The solvent may be a polar solvent. The solvent may be miscible with water. The solvent may be immiscible with water. The solvent may be, for example but not limited to, dichloromethane, acetone, methanol, ethanol, acetonitrile, chloroform, tetrahydrofuran (THF), pyridine, N,N-dimethylformamide, hexane, or a combination thereof. The solvent may be water.

The amount of solvent added may be enough to cover the powder in a container (e.g., a beaker, flask, or other glassware).

The pigment may be extracted from the fungal biomass by stirring the powdered fungus in the solvent directly. The powdered fungus or fungi and solvent may be stirred for a period of time. The stirring may be for about a half hour or more, about an hour or more, about 3 hours or more, about 5 hours or more, about 8 hours or more, or about 12 hours or more. The stirring may be continuous or generally continuous. For example, a stirring bar (e.g., a magnetic stirring bar) may be placed within the mixture. The speed of stirring may be sufficiently fast enough to mix the powdered fungus with the solvent. The speed of stirring may be selected so as not to cause splashing.

The stirring may take place under certain conditions. The stirring may take place at ambient temperature. The stirring may take place at a temperature greater than ambient temperature. The stirring may take place at a temperature that does not burn the solvent or powder. The stirring may take place at a temperature above the boiling point of the solvent. The stirring may take place at a temperature below the boiling point of the solvent. The stirring may take place while exposed to light (e.g., UV light). The stirring may take place in the absence of exposure to UV light.

The container may be open during stirring. The container may have a cap, plug, stopper, or other cover. The cover may be air permeable. The cover may be air impermeable. The cover may include one or more ventilation ports or openings for ventilation. The cover may be present to reduce or prevent evaporation of a solvent. A cover may be absent to encourage evaporation or partial evaporation of a solvent. A cover may be positioned at the opening of the container but not secured therein, thereby allowing for at least partial venting.

Following the stirring, a suitable filtering and/or separation operation may be conducted. Filtration may be performed, for example, using filter paper. A filter or filter paper may be placed at the opening of a beaker, flask, or container, and the solvent and powder may be poured onto the filter or filter paper. Solids of a size greater than the pores of the filter may be kept from entering the new container. Following filtration to filter out residue, a crude pigment extract in solvent may be present in the beaker, flask, or container.

Alternatively to open-container stirring, it is contemplated that pigment may be extracted from the fungus using a closed-loop system. For example, the pigment may be extracted from the fungal biomass in a closed-loop system using Soxhlet extraction. In the Soxhlet method, the fungal biomass may be contained within an extraction thimble and in a separate chamber from what is being stirred (e.g., solvent without a fungal biomass). In contrast from an open-container method, where the fungal biomass is submerged and stirred directly in the solvent, in the Soxhlet system, the fungal biomass would not be stirred. Using the Soxhlet method, it is possible that the majority or all of the solvent is not lost to evaporation during the process.

One or more purification steps may occur to produce a pure pigment. Any purification step that is capable of producing the desired purification of the pigment may be used. Thin-layer chromatography (TLC) may be used to determine an elution mixture having sufficient performance. TLC may be used to identify other substances present. The pigment or target compound may be purified using Column Chromatography, for example. A purification step may produce pure pigment in solvent.

Following purification, one or more evaporation steps may be performed. Evaporation steps may include, but are not limited to, open air or using a rotary evaporator. Following purification, the pure pigment (with or without prior evaporation steps) may be stirred into oil. Evaporation may occur after the pure pigment and solvent have been stirred into the oil.

Pigment may be produced from the fungus or fungi (e.g., extracted from the fungus or fungi). Pigments may be supplied as already-extracted fungal pigments (e.g., obtained from a third party). Pigments used may be selected from one or more categories, including but not limited to, isoprenoids, polyketides, quinones, naphthoquinones, flavins, pyrans, pyran-related, pyrones, lactiporic acids, and other non-proteins.

Pigments may include, but are not limited to, the following in Table 2, alone or in any combination:

TABLE 2
Pigment Names

2-dehydro-3-deoxylaetiporic acid A
5-chlorodermorubin
Acetylazulene
Albidin
Aleuriaxanthin
Alterperylenol
Amitenone
Ankaflavin
Anthraquinone
Arpink Red
Asperflavin-ribofuranoside
Aspergiolide A
Asperversin
Asperxanthin
Asp-melanin
Astaxanthin
Atromentin
Aurantricholide B
Aurasperone A
Aurosporin
Averythrin
Averythrin-6-monomethylether
Azaphilone
Azulene
Benzoquinone
Beta-carotene
Bikaverin
Bostrycoidin
Boviquinone
Canthaxanthin
Catenarin
Cercosporin
Chrysogenin
Chrysophanol
Citrinin
Citrosporin
Cochliodinol
Cordycepin
Cordycepoid A
Cynodontin
Dermocybin
Dermorubin
DHN-melanin
Dihydroxyazulene
DOPA-melanin
Draconin
Elsinochrome
Emodin
Epicocconone
Ergoxanthin
Ermolutein
Erythroglaucin
Eumelanin
Flavoglaucin
Flavomannin
Floccosin
Fonsecin
Furanones
Fusarubin
Gamma-carotene
GHB-melanin
Glauconic acid
Gomphidic acid
Grevilline A
Grifolinone
Haematopodin
Helminthosporin
Hydroxyanthraquinoid
Hypericin-like
Indigotin
Indigotine
Indirubin
Iridosporin
Isatin
Isodihydroauroglaucin
Javanicin
Juglone
Karuquinone A
Lactaroviolin
Laetiporic acid A
Laetiporic acid B
Laetiporic acid C
Lilacinone
Lovastatin
Luteosporin
Lycopene
Melanin
Melanocrocin
Mevinolin
Mitorubrin
Monaphilone A
Monaphilone B
Monarubrin
Monascin
Monascoflavin
Monascorubramine
Monascorubrin
Monascorubrine
Monascus-like
Monascuspilion
Monasfluore A
Mycenaaurin A
Mycosporine
Naphtoquinone
Nectriachrysone
Pachybasin
Parietin
Pestalone
Phallacinol
Phenoxazine
Pheomelanin
Phlegmacin A
Phomaligin A
Phomarin
Physcion
Pulvinone
Purpuride
Pyocyanin
Pyrandione
Pyrrocidine A
Questin
Riboflavin
Rubellin D
Rubosporin
Rubrocristin
Rubropunctamine
Rubropunctatin
Rubropunctatine
Rubropunctin
Rubrosulphin
Sanguinolentaquinone
Sanguinone A
Sanguinone B
Skyrin
Sorbicillin
Spinulosin
Stearoyldeterrol
Sterigmatocystin
Torularhodin
Torulene
Tritisporin
Tryptanthrin
Usnic acid
Variegatorubin
Viocristin
Violacein
Violosporin
Viomellein
Viopurpurin
Vioxanthin
Xanthomegnin
Xylindein

It is understood that certain pigments from natural sources are not yet named. These unnamed pigments may still be used within or as a colorant. For example, unnamed pigments produced from Scytalidium ganodermophthorum may produce a purple color or a yellow-olive color. In another example, Penicillium fagi may produce a greenish-blue color. Curvularia lunata may produce a purple color. Trichoderma virens may produce a purple color. Isaria spp. may produce a pink color. Emericella spp. may produce a pink color.

Pigment in solvent may be added to another medium. The other medium may be an oil. The oil may act as a carrier oil. The oil may ease or allow for mixing of the pigment within a cosmetic product. The oil may assist in stabilizing the color of the pigment (e.g., may keep the color from oxidizing). The oil may act to provide moisturizing, anti-aging, and/or restorative benefits to the cosmetic product.

While referred to herein as an oil, it is contemplated that the medium may be derived from oils. For example, vegetable glycerin is made from plant oils and may be a suitable medium. A medium derived from oil, such as vegetable glycerin, may be particularly useful if water is used as the solvent with the pigment.

The oil may be any of the following oils or any combination of any of the following oils. The oil may be raw oil. The oil may be natural oil. The oil may be synthetic oil. The oil may be hydrogenated. The oil may be partially hydrogenated. The oil may be an oil rich in fatty acids. The oil may be created by a fractionation process. For example, the oil may be medium-chain triglyceride oil (MCT oil).

The oil may have a smoke point of about 100° C. or greater, about 150° C. or greater, about 175° C. or greater, about 200° C. or greater, about 215° C. or greater, about 230° C. or greater, about 240° C. or greater, or about 250° C. or greater.

The oil may be a vegetable oil. Exemplary vegetable oils include crambe oil, rapeseed oil, corn oil, soybean oil, milkweed oil, coconut oil, palm oil, sunflower oil, cottonseed oil, lesquerella oil, canola oil, olive oil, peanut oil, avocado oil, rice bran oil, poppy oil, the like, or any combination thereof. The oil may be a seed oil. Exemplary seed oils include almond oil, argan oil, borage oil, castor oil, cherry pit oil, coconut oil, corn oil, cottonseed oil, linseed oil, grape seed oil, hemp oil, jojoba oil, macadamia oil, mango butter, mustard oil, neem oil, palm kernel oil, rapeseed oil, canola oil, safflower oil, sesame oil, shea butter, sunflower oil, tonka bean oil, tung oil, the like, or any combination thereof. The oil may be a nut oil. Exemplary nut oils include walnut oil, hazelnut oil, pecan oil, pistachio oil, peanut oil, almond oil, Brazil nut oil, the like, or any combination thereof. The nut oils may be roasted nut oils. The oil may be a wood finishing oil, such as Danish oil. Danish oil may include tung oil or polymerized linseed oil.

Enough pigment in solvent is added to the oil to produce the desired color within the oil, within the final product (e.g., a cosmetic), or both. The solvent may then be volatilized or evaporated.

One or more processes for evaporating the solvent may be used. The pigment may be isolated from the solvent using various methods of evaporation, including but not limited to rotary evaporation, nitrogen evaporation, freeze drying, spontaneous evaporation in the open air, the like, or a combination thereof. For example, a rotary evaporator may be used to remove the solvent from the oil and pigment. This may create a closed-loop solvent system that allows for work with particular solvents without wasting or releasing to the environment.

The oil and pigment in solvent may be stirred and/or heated to cause evaporation of the solvent. Stirring may occur at a speed that does not cause splashing within the container. Stirring may be generally continuous. For example, a magnetic stirring bar may be used. Stirring may occur for as long as necessary until the solvent evaporates or at least partially evaporates. Heating may expedite or encourage the evaporation. A temperature should be chosen that does not burn the pigment or the oil but allows for the solvent to evaporate. Stirring may occur until the consistency of the mixture is generally the same as that of the oil.

Upon evaporation of the solvent, the oil and pure pigment mixture may be used to provide color for a cosmetic product.

The present teachings contemplate a single fungus and/or a single pigment type, a single solvent, and a single oil. The present teachings contemplate two or more fungi, two or more pigments, two or more solvents, two or more oils, or a combination thereof. Where two or more fungi are used, the fungi may be mixed together prior to addition of a solvent. Where two or more fungi are used, mixing may occur at another point in the process. For example, each fungus may be solubilized in a solvent, thereby producing multiple samples of pure pigment in solvent. The pigments in solvents may be mixed together, prior to or after mixing with an oil. Such mixing may be desirable to produce a particular color. For example, mixing of blue pigments with yellow pigments may produce a green color. Additional oils may be added to a pigment in solvent. Additional oils may act to provide stability to the color. For example, a red pigment may be mixed with Danish oil. Adding almond oil, castor oil, and/or raw linseed oil (with or without additional pigment) may act to stabilize that color from oxidizing. This may result in a dark red-purple color that is very concentrated and very stable.

The colorant or the cosmetic product itself may include one or more components for providing shimmer, sheen, sparkle, pearlescence, or luster. The component may be synthetic. The component may be natural. The component may be derived from minerals. The component may be derived from crystals. For example, natural mica may be included in the composition. The component may include cellulose nanocrystals. Natural pearl luster pigments may include guanine and/or other heterocyclic compounds containing nitrogen in lamellar film. Pearl essences may include concentrated suspensions of crystals derived from fish scales, such as from herrings and sardines. Other natural products for providing luster include ground mollusk shells. These components may be added at any step of the process. For example, crystals derived from fish scales or mollusk shells may be ground or put into a fine powder or smaller and mixed with the powdered fungi; may be added to or with the oil; may be added after the oil; or may be mixed with the cosmetic base.

The colorant (e.g., pigment in oil after evaporation of the solvent) may be incorporated into a cosmetic base for providing color to the product. The cosmetic base may be oil-based. The cosmetic base may be water-based. The cosmetic base may include one or more oils, one or more waxes, or a combination thereof. The cosmetic base may include one or more of an emollient, an emulsifier, an anti-oxidant, or a combination thereof. The base may, for example, include one or more of candelilla wax, beeswax, MCT oil, castor oil, carnauba wax, vitamin E oil, tocopherol, almond oil, kaolin clay, jojoba oil, lanolin cera, shea butter, capryloyl glycerin/sebacic acid copolymer, caprylic/capric triglyceride, soybean oil, xanthan gum, avocado oil, coconut oil, mica, titanium dioxide, iron oxide, tin oxide, zinc oxide, powder, and cornstarch.

The colorant may be mixed with the base to provide the desired color of the cosmetic product. The cosmetic product may then be packaged according to the intended product or use. For example, a lipstick may be shaped using a mold and may be positioned within the tube for final use. A tinted moisturizer or sunscreen may be put into a bottle, jar, can, or squeezeable package, for example. An eyeshadow may be put into a pot, palette, clamshell container, tube, or the like.

It is contemplated that the cosmetic product may include substances other than the base for providing a desired effect. For example, active ingredients may be added to provide sun protection, acne treatment or prevention, anti-aging properties, replenishment of skin cells, turnover of skin cells, healing effects, changes to skin tone or texture, moisturizing, the like, or a combination thereof.

It should be understood that many additional changes in the details, materials, steps and arrangements of parts, which have been herein described and illustrated in order to explain the nature of the present invention, may be made by those skilled in the art while still remaining within the principles and scope of the invention.

As used herein, unless otherwise stated, the teachings envision that any member of a genus (list) may be excluded from the genus; and/or any member of a Markush grouping may be excluded from the grouping.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the comparative teaching of amounts expressed as weight/volume percent for two or more ingredients also encompasses relative weight proportions of the two or more ingredients to each other, even if not expressly stated. For example, if a teaching recites 2% A, and 5% B, then the teaching also encompasses a weight ratio of A:B of 2:5. Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference in their entirety for all purposes.

By use of the term “may” herein, it is intended that any described attributes that “may” be included are optional.

The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of (namely, the presence of any additional elements, ingredients, components or steps, does not materially affect the properties and/or benefits derived from the teachings); or even consist of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. All references herein to elements or metals belonging to a certain Group refer to the Periodic Table of the Elements published and copyrighted by CRC Press, Inc., 1989. Any reference to the Group or Groups shall be to the Group or Groups as reflected in this Periodic Table of the Elements using the IUPAC system for numbering groups.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

1. A colorant comprising:
a. one or more pigments derived from one or more fungi.

2. The colorant of claim 1, wherein the colorant is used for coloring a cosmetic product.

3. The colorant of claim 1, wherein the one or more pigments are extracted from the one or more fungi using a solvent or supercritical fluid.

4. The colorant of claim 3, wherein the solvent is dichloromethane, water, acetone, methanol, ethanol, acetonitrile, chloroform, tetrahydrofuran (THF), pyridine, N,N-dimethylformamide, hexane, or a combination thereof.

5. The colorant of claim 1, comprising one or more oils.

6. The colorant of claim 5, wherein the one or more oils comprise castor oil, almond oil, sunflower oil, walnut oil, linseed oil, Danish oil, MCT oil, vegetable glycerin, or a combination thereof.

7. The colorant of claim 1, wherein the colorant comprises pigment from mushroom, lichen, mold, yeast, or a combination thereof.

8. The colorant of claim 1, wherein the one or more fungi are spalting fungi.

9. The colorant of claim 1, wherein the one or more pigments are selected from one or more of: isoprenoids, polyketides, quinones, naphthoquinones, flavins, pyrans, pyran-related, pyrones, laetiporic acids, or other non-proteins.

10. The colorant of claim 1, wherein the one or more pigments include pigments of Table 2, alone or in any combination.

11. The colorant of claim 1, wherein the one or more pigments include beta carotene, melanin, astaxanthin, xylindein, draconin, unnamed yellow/purple pigment, monascus pigments, parietin, or a combination thereof.

12. The colorant of claim 1, wherein the one or more fungi for producing pigments of desirable colors include in any combination: Chlorociboria aeruginascens (Xylindein; blue-green), Chlorociboria aeruginosa (Xylindein; blue-green), Scytalidium cuboideum (Draconin; red), Monascus purpureus (Rubropunctatine, Monascorubrine, Monascoflavin, Lovastatin (mevinolin; orange), Monascus purpureus (Ankaflavin, Mosascin; yellow), Scytalidium ganodermophthorum (unknown pigment name; yellow, olive), Scytalidium ganodermophthorum (unknown pigment name; purple), Inonotus obliquus (Eumelanin; brown/black), Inonotus obliquus (Pheomelanin; red/yellow), Xanthoria parietina (Parietin; orange), Xanthophyllomyces dendrorhous (Astaxanthin; orange), Blakeslea trispora (Beta-carotene; yellow).

13.-28. (canceled)

29. The colorant of claim 2, wherein the cosmetic product is a lipstick, eyeliner, eyeshadow, mascara, blush, bronzer, moisturizer, nail polish, foundation, self-tanner, sunscreen, lotion, hair dye, shampoo, or conditioner.

30. The colorant of claim 2, wherein the cosmetic product is a hair product.

31. The colorant of claim 2, wherein the cosmetic product is a nail product.

32. The colorant of claim 1, wherein the one or more pigments derived from the one or more fungi is selected from the genus list consisting of: Monascus spp., Fusarium spp., Xanthophyllomyces spp., Trichoderma spp., Scytalidium spp., Inonotus spp., Aspergillus spp., Chlorociboria spp., Xanthoria spp.

33. The colorant of claim 1, further comprising one or more oils, wherein the one or more oils comprise at least almond oil.

34. The colorant of claim 1, wherein the colorant produces one of: a blue-green color, a red color, an orange color, a red color, a pink color, a purple color, a brown color, or a black color.

35. A cosmetic composition comprising:
one or more pigments derived from one or more fungi, wherein the fungi is selected from the genus list consisting of a genus: Monascus spp., Fusarium spp., Xanthophyllomyces spp., Trichoderma spp., Scytalidium spp., Inonotus spp., Aspergillus spp., Chlorociboria spp., Xanthoria spp.; and
an oil at least comprising almond oil.

36. The cosmetic composition of claim 35, wherein the cosmetic composition comprises a lipstick.
